(19) United States
(12) Patent Application Publication
(10) Pub. No.: US 2023/0139476 A1
(43) Pub. Date: May 4, 2023

(54) INFORMATION PROCESSING APPARATUS, INFORMATION PROCESSING METHOD, PROGRAM, AND OPHTHALMIC MICROSCOPE SYSTEM

(71) Applicant: SONY GROUP CORPORATION, TOKYO (JP)

(72) Inventor: YOSHIO SOMA, TOKYO (JP)

(21) Appl. No.: 17/906,702

(22) PCT Filed: Mar. 10, 2021
(86) PCT No.: PCT/JP2021/009523

§ 371 Date: Sep. 19, 2022

(30) Foreign Application Priority Data
Mar. 30, 2020 (JP) 2020-061541

(51) Int. Cl. A61B 3/135; A61B 3/00; A61B 3/14; G16H 30/40

(52) U.S. Cl. CPC A61B 3/135; A61B 3/0008; A61B 3/14; A61B 3/0058; G16H 30/40

(57) ABSTRACT

An information processing apparatus according to an embodiment of the present technology includes a generation unit. The generation unit generates difference information relating to a difference between a first observation condition that is an observation condition when observing an eye to be examined by a slit lamp microscope and a second observation condition that is an observation condition that is a basis with respect to observation of the eye to be examined by the slit lamp microscope. It is possible to easily perform operations in observation.

### TECHNICAL FIELD

The present technology relates to an information processing apparatus, an information processing method, a program, and an ophthalmic microscope system that can be applied to a slit lamp microscope.

### BACKGROUND ART

In an ophthalmic system described in Patent Literature 1, an ophthalmic imaging device including a slit lamp microscope acquires a three-dimensional image of an eye to be examined. Based on the acquired three-dimensional image, machine learning and data mining are performed and acknowledge is stored. Based on the stored acknowledge and the three-dimensional image of the eye to be examined, diagnosis assistance information is generated. Accordingly, analysis using artificial intelligence is favorably performed (paragraphs [0017], [0020], FIG. 8 and the like in Patent Literature 1).

### CITATION LIST

### Patent Literature

Patent Literature 1: Japanese Patent Application Laid-open No. 2019-24738

### DISCLOSURE OF INVENTION

### Technical Problem

In the slit lamp microscope, operations of an illumination unit and an imaging unit are performed manually. Therefore, it is difficult to reproduce conditions at the time of observation, such as an illumination direction and a camera position. It is thus desirable to provide a technology capable of easily performing operations in observation in a slit lamp microscope.

In view of the above-mentioned circumstances, it is an objective of the present technology to provide an information processing apparatus, an information processing method, a program, and an ophthalmic microscope system that are capable of easily performing operations in observation.

### Solution to Problem

In order to accomplish the above-mentioned objective, an information processing apparatus according to an embodiment of the present technology includes a generation unit.

The generation unit generates difference information relating to a difference between a first observation condition that is an observation condition when observing an eye to be examined by a slit lamp microscope and a second observation condition that is an observation condition that is a basis with respect to observation of the eye to be examined by the slit lamp microscope.

In this information processing apparatus, the difference information relating to the difference between the first observation condition that is the observation condition when observing the eye to be examined by the slit lamp microscope and the second observation condition that is the observation condition that is the basis with respect to the observation of the eye to be examined is generated. Accordingly, it is possible to easily perform operations in observation.

An information processing method according to an embodiment of the present technology is an information processing method that is executed by a computer system and includes generating difference information relating to a difference between a first observation condition that is an observation condition when observing an eye to be examined by a slit lamp microscope and a second observation condition that is an observation condition that is a basis with respect to observation of the eye to be examined by the slit lamp microscope.

A program according to an embodiment of the present technology causes a computer system to execute the following step.

A step of generating difference information relating to a difference between a first observation condition that is an observation condition when observing an eye to be examined by a slit lamp microscope and a second observation condition that is an observation condition that is a basis with respect to observation of the eye to be examined by the slit lamp microscope.

An ophthalmic microscope system according to an embodiment of the present technology includes a slit lamp microscope and an information processing apparatus.

The information processing apparatus includes a generation unit.

The generation unit generates difference information relating to a difference between a first observation condition that is an observation condition when observing an eye to be examined by a slit lamp microscope and a second observation condition that is an observation condition that is a basis with respect to observation of the eye to be examined by the slit lamp microscope.

### BRIEF DESCRIPTION OF DRAWINGS

FIG. 1 A schematic diagram for describing the overview of an observation system.

FIG. 2 A block diagram showing a functional configuration example of the observation system.

FIG. 3 A schematic diagram showing an example of image analysis.

FIG. 4 A flowchart showing an example of guide information generation.

FIG. 5 A schematic diagram showing an example of a guide display GUI.

FIG. 6 A schematic diagram showing another example of the guide display GUI.

FIG. 7 A flowchart showing an example of a procedure of imaging plan generation.

FIG. 8 A block diagram showing a hardware configuration example of an information processing apparatus.

### MODE(S) FOR CARRYING OUT THE INVENTION

Hereinafter, embodiments according to the present technology will be described with reference to the drawings.

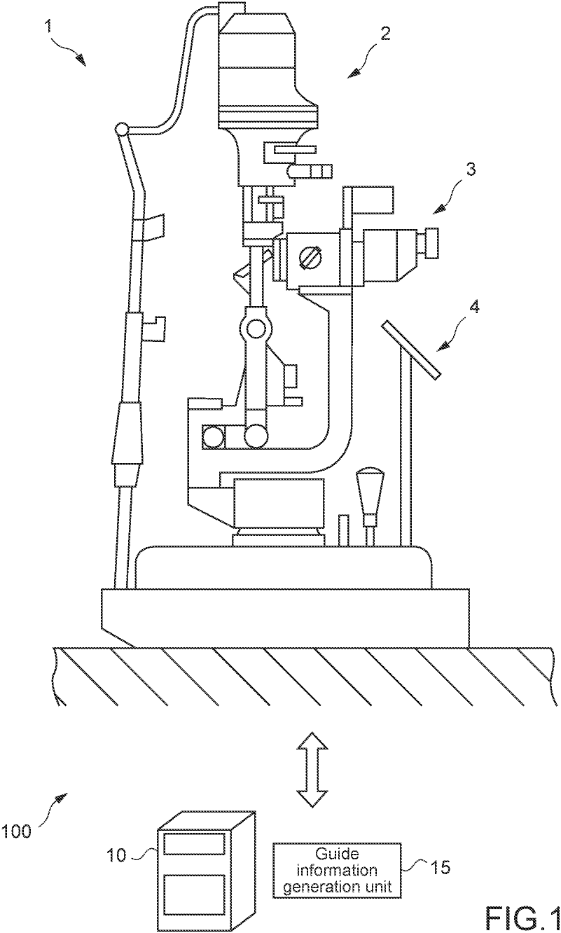

FIG. 1 is a schematic diagram for describing the overview of an observation system according to the present technology. It should be noted that an observation system 100 corresponds to an embodiment of an ophthalmic microscope system according to the present technology.

As shown in FIG. 1, the observation system 100 includes a slit lamp microscope 1 and an information processing apparatus 10.

The slit lamp microscope 1 and the information processing apparatus 10 are connected to one another via wires or wirelessly so that they can communicate with one another. The connection form between the respective devices is not limited. For example, it is possible to utilize wireless LAN communication such as Wi-Fi or near-field communication such as Bluetooth (registered trademark).

The slit lamp microscope 1 includes the illumination optical system 2 and the imaging optical system 3 and is capable of observing the eye to be examined. A user (e.g., a doctor) manually or electrically operates the illumination optical system 2 and the imaging optical system 3 to thereby observe the eye to be examined.

The illumination optical system 2 is capable of emitting slit light toward the eye to be examined.

The imaging optical system 3 is capable of imaging light reflected from the eye to be examined. For example, the imaging optical system includes a camera for the right eye and a camera for the left eye that are capable of imaging eyes to be examined.

It should be noted that specific configurations of the illumination optical system 2 and the imaging optical system 3 are not limited. For example, an image sensor such as a complementary metal-oxide semiconductor (CMOS) sensor and a charge coupled device (CCD) sensor may be used as an imaging device and an imaging element for imaging the eye to be examined.

In this embodiment, the slit lamp microscope 1 includes a display unit 4. On the display unit 4, difference information generated by the information processing apparatus 10 is presented.

It should be noted that a configuration of the slit lamp microscope 1 is not limited. For example, the slit lamp microscope 1 may include a drive mechanism or the like capable of changing the position of the display unit 4. Moreover, for example, the slit lamp microscope 1 does not need to include the display unit 4 and the difference information may be presented on a device such as a personal computer (PC).

The observation condition at least includes an illumination condition relating to the illumination optical system 2 included in the slit lamp microscope 1 and an imaging condition relating to the imaging optical system 3 included in the slit lamp microscope 1.

The illumination condition includes at least one of the position of slit light emitted to the eye to be examined, the position of the illumination optical system 2, the amount of light of the slit light, or the width (shape) of the slit light.

The imaging condition includes at least one of the position, the scale, or the imaging direction of the imaging optical system 3.

In this embodiment, the observation condition includes a current condition indicating a real-time condition when observing the eye to be examined by the slit lamp microscope 1 and a reference condition indicating a condition that is a basis with respect to observation of the eye to be examined by the slit lamp microscope 1. For example, an illumination condition to emit slit light in a predetermined direction and an imaging condition to image the eye to be examined in a predetermined direction are the reference condition.

The difference information is information indicating a difference between the observation conditions. In this embodiment, a difference between the current condition and the reference condition is generated as the difference information. For example, a difference between a current position of the illumination optical system 2 and a reference position of the illumination optical system 2 are generated as the difference information. Specifically, difference information of an error of 3 cm or the like from coordinates indicating the position of the illumination optical system 2 is generated.

The information processing apparatus 10 is capable of acquiring an observation condition of the slit lamp microscope 1 and generating difference information. In this embodiment, the information processing apparatus 10 presents the generated difference information on the display unit 4 mounted on the slit lamp microscope 1. For example, the information processing apparatus 10 causes the display unit 4 to display a graphical user interface (GUI) in which the difference information is displayed so as to be identifiable to the user.

It should be noted that in this embodiment, the current condition corresponds to a first observation condition that is an observation condition when observing an eye to be examined by a slit lamp microscope. The reference condition corresponds to a second observation condition that is an observation condition that is a basis with respect to observation of the eye to be examined by the slit lamp microscope.

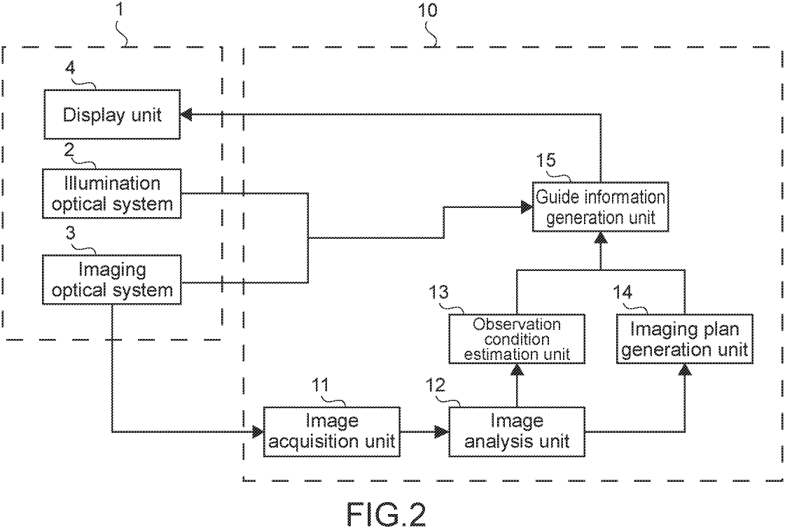

FIG. 2 is a block diagram showing a configuration example of the observation system 100.

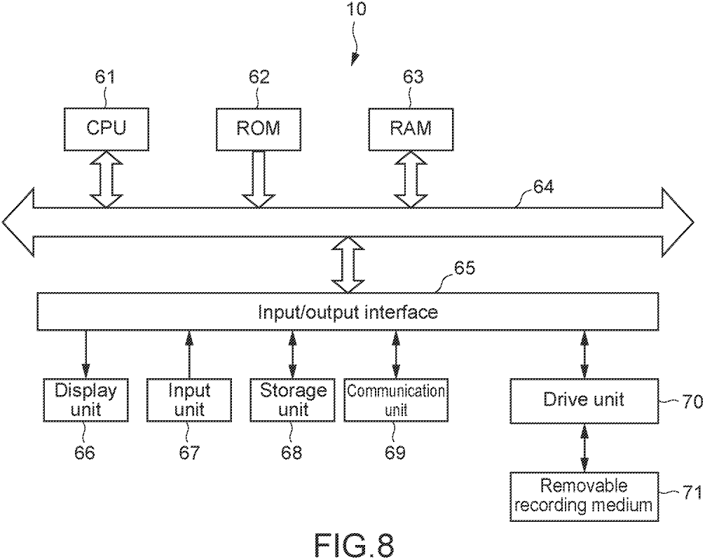

The information processing apparatus 10 includes hardware required for configurations of a computer including, for example, processors such as a CPU, a GPU, and a DSP, memories such as a ROM and a RAM, a storage device such as an HDD (see FIG. 8). For example, the CPU loads a program according to the present technology recorded in the ROM or the like in advance to the RAM and executes the program to thereby execute an information processing method according to the present technology.

For example, any computer such as a PC can realize the information processing apparatus 10. As a matter of course, hardware such as FPGA and ASIC may be used. In this embodiment, when the CPU executes a predetermined program, a guide information generation unit as a functional block is configured. As a matter of course, dedicated hardware such as an integrated circuit (IC) may be used for realizing functional blocks.

The program is, for example, installed in the information processing apparatus 10 via various recording media. Alternatively, the program may be installed via the Internet.

The kind of recording medium and the like in which the program is recorded are not limited, and any computer-readable recording medium may be used. For example, any computer-readable non-transitory storage medium may be used.

As shown in FIG. 2 the information processing apparatus includes an image acquisition unit 11, an image analysis unit 12, an observation condition estimation unit 13, an he imaging plan generation unit 14, and a guide information generation unit 15.

The image acquisition unit 11 acquires a captured image including the eye to be examined. In this embodiment, the image acquisition unit 11 acquires the captured image captured by the imaging optical system 3. That is, the captured image under the current imaging condition is captured and acquired by the image acquisition unit 11.

Further, in this embodiment, the image acquisition unit 11 acquires a reference image that is the captured image under the reference condition. It should be noted that a method of acquiring the reference image is not limited, and the slit lamp microscope 1 may set a captured image captured under a predetermined observation condition as the reference image. Moreover, for example, a reference image including a different eye to be examined (patient) may be externally acquired.

The acquired captured image and reference image are output to the image analysis unit 12.

The image analysis unit 12 analyzes the captured image and the reference image. For example, the image analysis unit 12 performs analysis by the image recognition, threshold processing, segmentation, image signal analysis, and the like. The analysis method is not limited, and any method may be used. For example, image analysis may be performed by machine learning.

Further, for example, the image analysis unit 12 is capable of recognizing the positions of the irises, blood vessel structures on the sclerae, the eyelids, and the like from the captured image and the reference image.

In this embodiment, a result of the analysis performed by the image analysis unit 12 are output to the observation condition estimation unit 13 and the imaging plan generation unit 14.

The observation condition estimation unit 13 estimates an observation condition. In this embodiment, the observation condition estimation unit 13 estimates the observation condition on the basis of the analysis result.

For example, on the basis of an eyeball positional relationship of the irises and the like, the position of the imaging optical system 3 is estimated. Moreover, for example, on the basis of feature extraction, Hough transform, and the like of the captured image, an imaging direction and a scale of the imaging optical system 3 are estimated. Moreover, for example, on the basis of image signals of the captured image, the aperture of the imaging optical system 3, the f-number, the color of the lens (or acquired by the sensor), exposure to light, or the shutter speed are estimated.

For example, on the basis of the image signals of the captured image, the amount of light of the slit light emitted from the illumination optical system 2, the wavelength, and the presence/absence or kind of filter are estimated. Moreover, for example, on the basis of the threshold processing of the captured image, the illumination direction of the illumination optical system 2 and the shape (width or angle) of the slit light are estimated. Moreover, for example, on the basis of the image recognition of the captured image, an observation technique such as diaphanoscopy is estimated.

Further, in this embodiment, the estimated current condition and reference condition are output to the guide information generation unit 15.

The imaging plan generation unit 14 generates imaging plan for collecting training data. In this embodiment, the imaging plan is generated on the basis of a learning algorithm that the user wishes to make and the number of captured images that the user specifies.

The imaging plan is an observation condition for acquiring a captured image that satisfies training data of the learning algorithm that the user specifies.

For example, it is assumed that the user has specified a learning algorithm capable of determining whether or not the eye to be examined is suffering from cataract with hundred captured images. In this case, the imaging plan generation unit 14 generates an imaging plan to image ten captured images under each of observation conditions under which a predetermined angle and a predetermined amount of light are set, using an eye to be examined suffering from cataract as a target.

The guide information generation unit 15 generates guide information including the difference information and the imaging plan. For example, the guide information generation unit 15 generates the difference information on the basis of an estimation result output from the observation condition estimation unit 13.

In this embodiment, the guide information generation unit 15 causes the display unit 4 to display a GUI in which the difference information is displayed so as to be identifiable to the user.

Further, in this embodiment, the guide information generation unit 15 causes the display unit 4 to display a GUI in which the imaging plan is displayed so as to be identifiable to the user.

It should be noted that a method of generating the guide information is not limited. For example, observation values corresponding to the observation conditions of the illumination optical system 2 and the imaging optical system 3 may be acquired from the slit lamp microscope 1. Specifically, the difference information is generated on the basis of a difference between an observation value indicating coordinates of the imaging optical system 3, which corresponds to the current condition, and an observation value indicating coordinates of the imaging optical system 4, which corresponds to the reference condition.

It should be noted that in this embodiment, the guide information generation unit 15 corresponds to a generation unit that generates difference information relating to a difference between a first observation condition that is an observation condition when observing an eye to be examined by a slit lamp microscope and a second observation condition that is an observation condition that is a basis with respect to observation of the eye to be examined by the slit lamp microscope.

It should be noted that in this embodiment, the observation condition estimation unit 13 corresponds to an estimation unit that estimates the observation condition relating to the slit lamp microscope on the basis of a captured image including the eye to be examined.

It should be noted that in this embodiment, the guide information generation unit 15 and the display unit 4 function as a presentation unit that presents the difference information to a user.

It should be noted that in this embodiment, the imaging plan generation unit 14 corresponds to a plan generation unit that generates an imaging plan for acquiring the captured image as training data to be used for machine learning.

It should be noted that in this embodiment, the display unit 4 corresponds to an image display unit included in the slit lamp microscope.

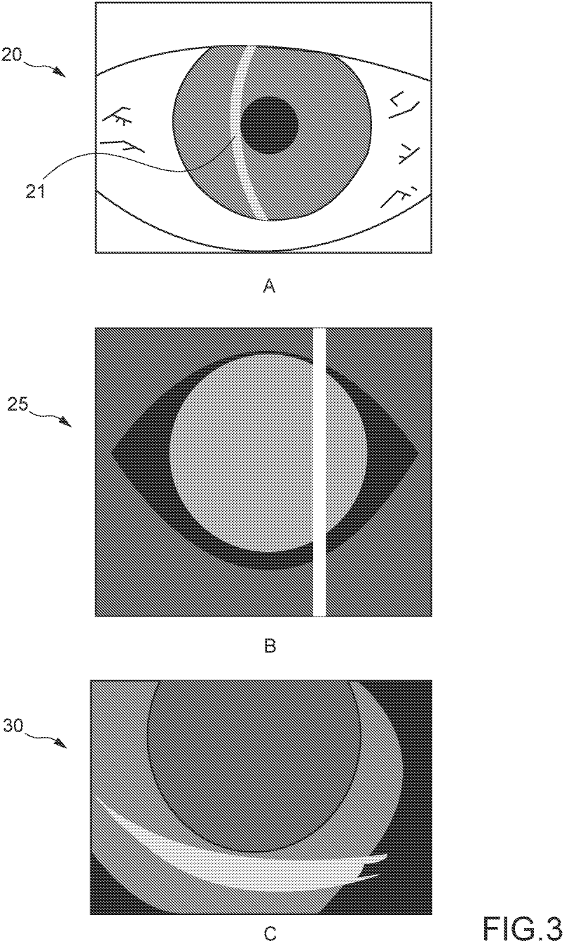

FIG. 3 is a schematic diagram showing an example of the image analysis. FIG. 3 shows FIGS. 3A to C as examples of images analyzed by the image analysis unit 12.

FIG. 3A is a schematic diagram of an image in a state in which slit light is emitted to the eye to be examined.

As shown in FIG. 3A, slit light 21 is emitted to an eye to be examined 20. The image analysis unit 12 analyzes the image signals of the captured image, and the observation condition estimation unit 13 can thus estimate the amount of light of the emitted slit light, the position of the illumination optical system 2, and the position of the imaging optical system 3.

FIG. 3B is a schematic diagram of an image in a state in which the eye to be examined is observed by diaphanoscopy.

For example, the image analysis unit 12 may analyze that an eye to be examined 25 in FIG. 3B is being observed by diaphanoscopy by machine learning.

FIG. 3C is a schematic diagram of an image in a state in which fluorescence is emitted from the illumination optical system 2.

In FIG. 3C, fluorescein is applied to the eye to be examined 30. For example, the image analysis unit 12 is capable of analyzing the fact that fluorescein has been used on the basis of the color or the like and the fact that light having a wavelength corresponding to fluorescence has been emitted from the illumination optical system 2.

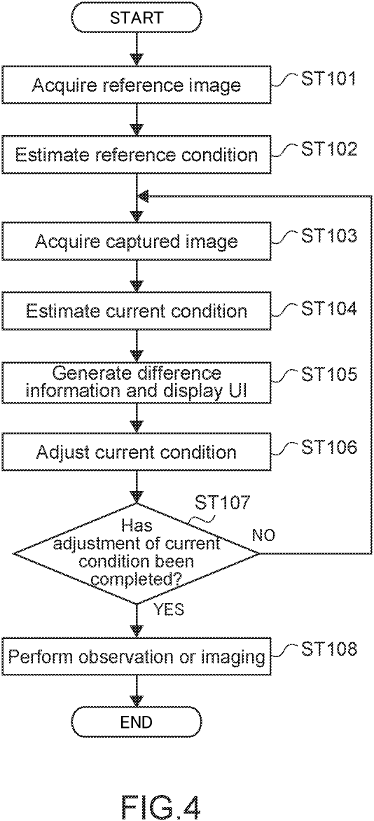

FIG. 4 is a flowchart showing an example of the guide information generation.

In a case where the user wishes to take a captured image under a predetermined condition, the image acquisition unit 11 acquires a reference image that satisfies a predetermined condition (Step 101). For example, it is assumed that the user wishes to take a captured image captured from the front by emitting slit light to the eye to be examined at a predetermined angle. In this case, the image acquisition unit 11 acquires a reference image that satisfies the condition.

A method of acquiring the reference image is not limited, and image recognition may be used with respect to the reference image and whether or not it satisfies the condition may be determined. Alternatively, the reference condition may be associated with the reference image and the reference image may be acquired by referring to the reference condition.

The image analysis unit 12 analyzes the reference image and the observation condition estimation unit 13 estimates the reference condition (Step 102).

The image acquisition unit 11 acquires a captured image captured by the slit lamp microscope 1 (Step 103). The observation condition estimation unit 13 estimates a current condition from the acquired captured image (Step 104).

The guide information generation unit 15 generates a difference information on the basis of the estimated reference condition and current condition. Moreover, a GUI in which the difference information is displayed so as to be identifiable to the user is displayed on the display unit 4 (Step 105).

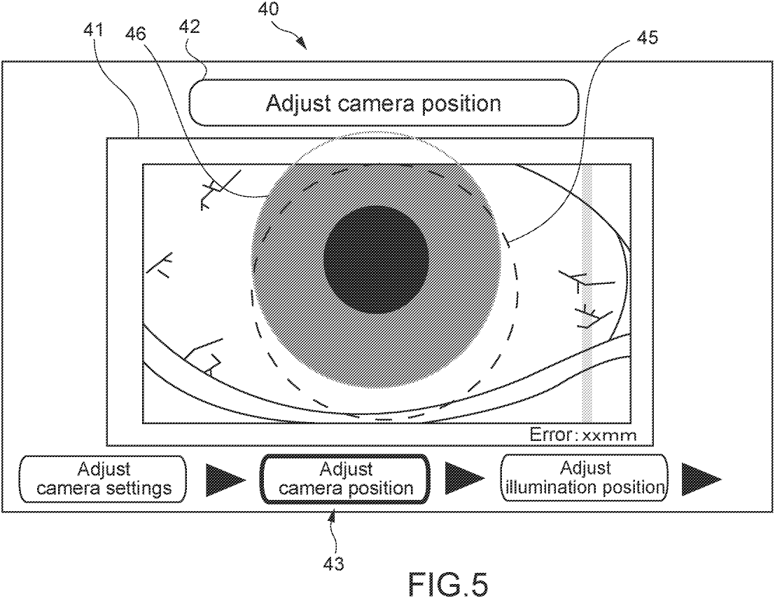

FIG. 5 is a schematic diagram showing an example of a guide display GUI.

As shown in FIG. 5, a guide display GUI 40 includes an image display unit 41, a guide display unit 42, and a chart display unit 43. In this embodiment, guide information and guide text are displayed on the guide display GUI 40 as the difference information.

The image display unit 41 displays the captured image captured by the slit lamp microscope 1 and the guide information. As shown in FIG. 5, the guide information (dotted line 45) are shown on the image display unit 41. In FIG. 5, the dotted line 45 indicates the outline of the iris of the reference image. That is, by adjusting an outline 46 of the iris of the captured image to the dotted line 45, it is possible to adjust the observation condition of the imaging optical system 3 to the reference condition.

In this embodiment, the image display unit 41 displays the guide text. For example, a distance between the current center of the pupil of the eye to be examined and the center of the dotted line 45 is displayed as the guide text “error: xx mm”.

The guide display unit 42 displays a guide text for matching the current condition to the reference condition. For example, in FIG. 5, the guide text “adjust the camera position” for matching the position of the camera (imaging optical system 3) to the reference condition is displayed on the guide display unit 42.

The guide text displayed on the guide display unit 42 is displayed with a chart of the chart display unit 43.

The chart display unit 43 displays a chart for matching the current condition to the reference condition. In FIG. 5, “camera setting adjustment”, “camera adjustment”, and “illumination adjustment” are displayed as the chart. Moreover, in FIG. 5, the “camera adjustment” has been performed and the frame of the “camera adjustment” is displayed as the thick lines. Accordingly, the user can easily know which condition of the observation conditions should be matched.

Further, the chart display unit 43 newly displays a chart in a case where the displayed chart has been completed. In a case where all conditions of the current conditions are matched to the reference conditions, the display of the chart display unit 43 is completed.

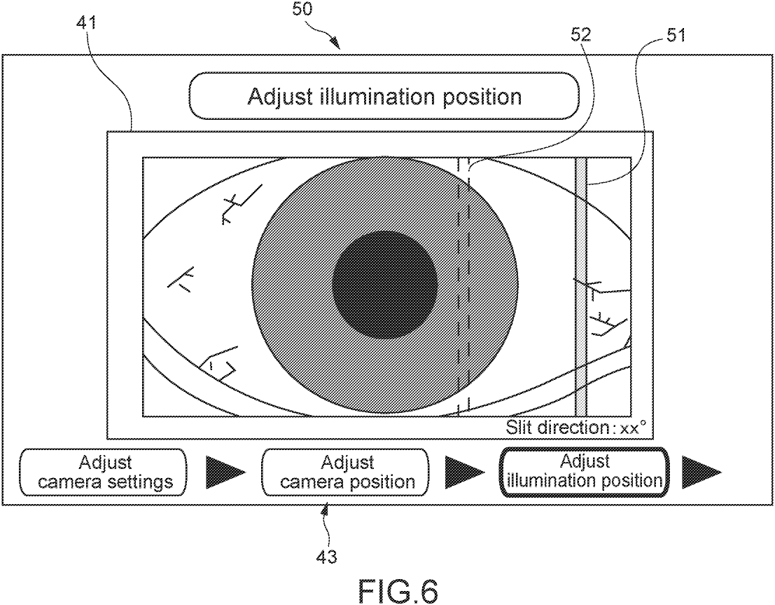

FIG. 6 is a schematic diagram showing another example of the guide display GUI.

in FIG. 6, a guide display GUI 50 is a GUI in a state in which the chart of the guide display GUI 40 in FIG. 5 has progressed. That is, this is the GUI at a stage at which the chart of the “camera adjustment” has been completed and the chart of the “illumination adjustment” is to be performed.

As shown in FIG. 6, the image display unit 41 displays guide information (dotted line 52) for adjusting a current illumination position 51 to a reference illumination position. Moreover, the image display unit 41 displays a difference between the current position of the slit light and the position of the dotted line 52 as the guide text “slit direction: xx degrees”.

It should be noted that a method of presenting the difference information is not limited. For example, the guide text, e.g., “move the camera by xx mm” may be presented by sound. Moreover, a configuration of the guide display GUI is not limited, and the user may be able to arbitrarily set it.

The user adjusts the current condition to match the reference condition in accordance with the guide text in FIGS. 5 and 6 (Step 106). In a case where the user has completed the adjustment of the current condition (YES in Step 107), the user can perform imaging (observation) under a desired reference condition (Step 108).

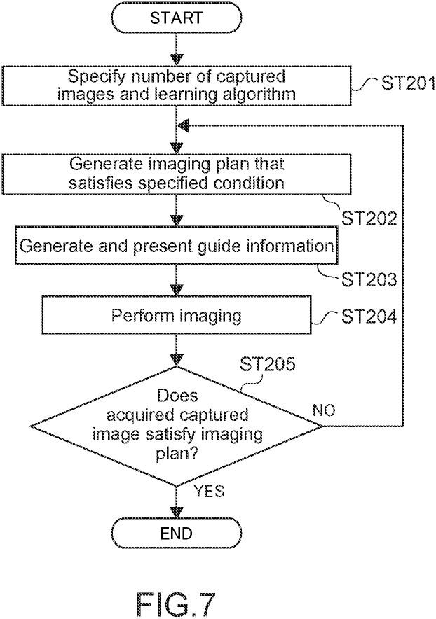

FIG. 7 is a flowchart showing an example of a procedure of the imaging plan generation.

The user specifies a desired learning algorithm and the number of captured images that is training data for generating the learning algorithm (Step 201).

The imaging plan generation unit 14 generates imaging plan that satisfies the specified condition (Step 202). In this embodiment, the imaging plan generation unit 14 generates imaging plan to have a sufficient distribution with respect to the specified condition. For example, an imaging plan that to image the eye to be examined with small and large, various amounts of light as the amount of light of slit light at any angle is generated.

The guide information generation unit 15 generates the generated imaging plan as the guide information and causes the display unit 4 to display it (Step 203). For example, like the guide display GUI 40 shown in FIG. 5, the GUI for matching the observation condition to the current condition included in the imaging plan may be displayed on the display unit 4. Moreover, for example, the imaging plan may be presented to the user by sound.

The user performs imaging in accordance with the imaging plan (Step 204). Whether or not the captured image acquired by the imaging plan generation unit 14 satisfies the imaging plan is determined (Step 205). In a case where the acquired captured image is not sufficient as training data for the imaging plan (NO in Step 205), an imaging plan for acquiring new training data is newly generated (Step 202). Accordingly, it is possible to efficiently generate training data for the machine learning.

Hereinabove, in the observation system 100 according to this embodiment, difference information relating to a difference between a first observation condition that is an observation condition when observing the eye to be examined by the slit lamp microscope 1 and a second observation condition that is an observation condition that is a basis with respect to observation of the eye to be examined by the slit lamp microscope 1 is generated. Accordingly, it is possible to easily perform operations in observation.

In general, in an examination or diagnosis based on observation or images, observed images or obtained images change depending on various conditions. For obtaining quantitative, reproducible results, observation or images under a uniform condition where the conditions are as uniform as possible are desirable. It is more important especially in a case of an examination or diagnosis where comparison is performed like a follow-up examination because the focus is put only on a change in lesioned part.

Further, also in a diagnosis using artificial intelligence (AI) using images, an acquisition condition of images is important. The same applies both to the time of learning when a machine learning model is generated and the time of utilization when a diagnosis is performed using the machine learning model. In the machine learning, it is desirable to uniformly include information acquired under various conditions at the time of learning. Moreover, it is desirable that in a case of using a learned model, an acquisition condition of images to be assessed be not different from an acquisition condition included in training data.

In view of this, in the present technology, in the use of the slit lamp microscope in which many manual interruptions are required (e.g., setting at the time of observation by the slit lamp microscope), difference information relating to a difference between a current condition and a condition that is a basis is generated in order to perform observation or image acquisition under the same condition. As a result, observation under the same condition as previous images becomes easy, and quantitative and reproducible examination and diagnosis become possible.

Further, since the difference information for matching to the condition that is the basis is presented, special skill is unnecessary for the slit lamp microscope and it is possible to easily and quickly set a condition at the time of observation. Moreover, since captured images under a predetermined condition can be acquired, training data for machine learning can be efficiently generated. In addition, when assessment is performed by machine learning, highly accurate examination and diagnosis can be performed under uniform conditions.

### Other Embodiments

The present technology is not limited to the above-mentioned embodiments, and various other embodiments can be realized.

In the above-mentioned embodiments, the training data is used as the method of generating the learning algorithm. The present technology is not limited thereto, and various learning algorithms and generation methods therefor may be used.

For example, an arbitrary machine learning algorithm using a deep neural network (DNN) or the like may be used. For example, by using artificial intelligence (AI) or the like that performs deep learning, generation of the learning algorithm can be improved.

For example, the learning unit and the identification unit are built for generating the learning algorithm. The learning unit performs machine learning on the basis of input information (learning data) and outputs the learning result. Moreover, the identification unit performs identification of the input information (e.g., judgement, prediction) on the basis of the input information and the learning result.

For example, neural network and deep learning are used for learning techniques in the learning unit. The neural network is a model that mimics neural networks of a human brain. The neural network is constituted by three types of layers of an input layer, an intermediate layer (hidden layer), and an output layer.

The deep learning is a model using neural networks with a multi-layer structure. The deep learning can repeat characteristic learning in each layer and learn complicated patterns hidden in mass data.

The deep learning is, for example, used for the purpose of identifying objects in an image or words in a speech. For example, a convolutional neural network (CNN) or the like used for recognition of an image or moving image is used.

Moreover, a neuro chip/neuromorphic chip in which the concept of the neural network has been incorporated can be used as a hardware structure that realizes such machine learning.

Supervised learning, unsupervised learning, semi-supervised learning, reinforcement learning, inverse reinforcement learning, active learning, transfer learning, and the like exist for problem settings in machine learning.

For example, supervised learning learns feature amounts on the basis of provided labeled learning data (training data). Accordingly, labels of unknown data can be derived.

Moreover, unsupervised learning analyzes a large amount of unlabeled learning data, extracts feature amounts, and performs clustering on the basis of the extracted feature amounts. Accordingly, trend analysis and future prediction can be performed on the basis of a huge amount of unknown data.

Moreover, semi-supervised learning is mixed supervised learning and unsupervised learning. The semi-supervised learning is a method in which feature amounts are learned in supervised learning, and then a large amount of training data is provided in unsupervised learning and learning is repeatedly performed while feature amounts are automatically computed.

Moreover, reinforcement learning handles a problem in that an agent in a certain environment observes a current state and determines an action that the agent should take. The agent selects an action to thereby get a reward from the environment and learns a policy that can maximize the reward through a series of actions. In this manner, learning an optimal solution in a certain environment can reproduce the human judgement ability and can also cause a computer to learn a judgement ability beyond the human judgement ability.

Virtual sensing data can also be generated by machine learning. It is possible to predict other sensing data from certain sensing data and uses it as the input information, for example, generate positional information from input image information.

Moreover, it is also possible to generate other sensing data from a plurality of pieces of sensing data. Moreover, it is also possible to predict necessary information and generate predetermined information from the sensing data.

In the above-mentioned embodiments, the slit lamp microscope 1 captures the captured image that is the training data necessary for the imaging plan specified by the user. The present technology is not limited thereto, and the captured image that satisfies the imaging plan may be arbitrarily acquired. For example, hundred captured images obtained by imaging the eye to be examined from the front may be acquired from another user and three hundred captured images obtained by imaging the eye to be examined at a predetermined angle may be acquired from still another user.

In the above-mentioned embodiments, the guide display GUI 40 is displayed on the display unit 4. The present technology is not limited thereto, and for example, the guide display GUI 40 may be presented to the user by looking into the eyepieces of the slit lamp microscope 1.

FIG. 8 is a block diagram showing a hardware configuration example of the information processing apparatus 10.

The information processing apparatus 10 includes a CPU 61, a ROM 62, a RAM 63, an input/output interface 65, and a bus 64 that connects them to one another. A display unit 66, an input unit 67, a storage unit 68, a communication unit 69, and a drive unit 70, and the like are connected to the input/output interface 65.

The display unit 66 is, for example, a display device using liquid-crystal, EL, or the like. The input unit 67 is, for example, a keyboard, a pointing device, a touch panel, or another operation device. In a case where the input unit 67 includes a touch panel, the touch panel can be integral with the display unit 66.

The storage unit 68 is a nonvolatile storage device and is, for example, an HDD, a flash memory, or another solid-state memory. The drive unit 70 is, for example, a device capable of driving a removable recording medium 71 such as an optical recording medium and a magnetic record tape.

The communication unit 69 is a modem, a router, or another communication device for communicating with the other devices, which are connectable to a LAN, WAN or the like. The communication unit 69 may perform wired communication or may perform wireless communication. The communication unit 69 is often used separately from the information processing apparatus 10.

The information processing by the information processing apparatus 10 having the hardware configuration as described above is realized by cooperation of software stored in the storage unit 68, the ROM 62, or the like with hardware resources of the information processing apparatus 10. Specifically, by loading the program that configures the software to the RAM 63, which has been stored in the ROM 62 or the like, and executing the program, the information processing method according to the present technology is realized.

The program is, for example, installed in the information processing apparatus 10 via the recording medium 71. Alternatively, the program may be installed in the information processing apparatus 10 via a global network or the like. Otherwise, any computer-readable non-transitory storage medium may be used.

By cooperation of a computer mounted on a communication terminal with another computer capable of communicating with it via a network or the like, the information processing apparatus, the information processing method, the program, and the ophthalmic microscope system according to the present technology may be executed and the information processing apparatus according to the present technology may be configured.

That is, the information processing apparatus, the information processing method, the program, and the ophthalmic microscope system according to the present technology can be executed not only in a computer system configured by a single computer but also in a computer system in which a plurality of computer operates in cooperation. It should be noted that in the present disclosure, the system means a group of a plurality of components (apparatuses, modules (components), and the like) and it does not matter whether or not all components is in the same casing. Therefore, a plurality of apparatuses housed in separate casings and connected via a network and a single apparatus in which a plurality of modules is housed in a single casing are both systems.

The execution of the information processing apparatus, the information processing method, the program, and the ophthalmic microscope system according to the present technology by the computer system includes, for example, both a case where estimating the observation condition, outputting the GUI, generating the imaging plan, and the like are performed by a single computer and a case where the respective processes are performed by different computers. Moreover, execution of the respective processes by a predetermined computer includes causing another computer to performing some or all of the processes to acquire the results.

That is, the information processing apparatus, the information processing method, the program, and the ophthalmic microscope system according to the present technology can also be applied to a cloud computing configuration in which a single function is shared and cooperatively processed by a plurality of apparatuses via a network.

The respective configurations such as the observation condition estimation unit, the imaging plan generation unit, and the guide information generation unit, the control flows of the communication system, and the like, which have been described with reference to the respective drawings, are merely embodiments, and can be arbitrarily modified without departing from the gist of the present technology. That is, any other configuration, algorithm, and the like for carrying out the present technology may be employed.

It should be noted that the effects described in the present disclosure are merely exemplary and not limitative, and also other effects may be provided. The above descriptions of the plurality of effects do not mean that those effects are always provided at the same time. They mean that at least any one of the above-mentioned effects is provided depending on a condition or the like. As a matter of course, effects not described in the present disclosure can be provided.

At least two feature parts of the feature parts of the above-mentioned embodiments can also be combined. That is, various feature parts described in each of the above-mentioned embodiments may be arbitrarily combined across those embodiments.

In the present disclosure, it is assumed that the concepts that define the shape, the size, the position relationship, the state, and the like such as “center”, “middle”, “uniform”, “equal”, the “same”, “orthogonal”, “parallel”, “symmetric”, “extending”, “axial”, “columnar”, “cylindrical”, “ring-shaped”, and “annular” are concepts including “substantially center”, “substantially middle”, “substantially uniform”, “substantially equal”, “substantially the same”, “substantially orthogonal”, “substantially parallel”, “substantially symmetric”, “substantially extending”, “substantially axial”, “substantially columnar”, “substantially cylindrical”, “substantially ring-shaped”, “substantially annular”, and the like.

For example, states included in a predetermined range (e.g., ±10% range) using “completely center”, “completely middle”, “completely uniform”, “completely equal”, “completely the same”, “completely orthogonal”, “completely parallel”, “completely symmetric”, “completely extending”, “completely axial”, “completely columnar”, “completely cylindrical”, “completely ring-shaped”, “completely annular”, and the like as the basis are also included.

It should be noted that the present technology can also take the following configurations.
(1) An information processing apparatus, including
   a generation unit that generates difference information relating to a difference between a first observation condition that is an observation condition when observing an eye to be examined by a slit lamp microscope and a second observation condition that is an observation condition that is a basis with respect to observation of the eye to be examined by the slit lamp microscope.
(2) The information processing apparatus according to (1), further including
   an estimation unit that estimates the observation condition on the basis of a captured image including the eye to be examined.
(3) The information processing apparatus according to (1) or (2), in which
   the observation condition at least includes an illumination condition relating to an illumination optical system included in the slit lamp microscope and an imaging condition relating to an imaging optical system included in the slit lamp microscope.
(4) The information processing apparatus according to (2), in which
   the estimation unit estimates the illumination condition on the basis of the captured image.
(5) The information processing apparatus according to (3), in which
   the illumination condition includes at least one of a position, an illumination direction, an amount of light, or a shape of illumination light.
(6) The information processing apparatus according to (2), in which
   the estimation unit estimates the imaging condition on the basis of the captured image.
(7) The information processing apparatus according to (3), in which the imaging condition includes at least one of a position, a scale, or an imaging direction.
(8) The information processing apparatus according to (3), in which
   the generation unit generates the difference information on the basis of a difference between a first illumination condition included in the first observation condition and a second illumination condition included in the second observation condition.
(9) The information processing apparatus according to (3), in which
   the generation unit generates the difference information on the basis of a difference between a first imaging condition included in the first observation condition and a second imaging condition included in the second observation condition.
(10) The information processing apparatus according to any one of (1) to (9), further including
   a presentation unit that presents the difference information to a user.
(11) The information processing apparatus according to (10), in which
   the presentation unit presents a graphical user interface (GUI) in which the difference information is displayed so as to be identifiable to the user.
(12) The information processing apparatus according to (10) or (11), in which
   the presentation unit presents the difference information to the user by sound.
(13) The information processing apparatus according to any one of (10) to (12), in which
   the slit lamp microscope includes an image display unit, and
   the presentation unit causes the image display unit to display the GUI.
(14) The information processing apparatus according to any one of (1) to (13), in which
   the generation unit generates the difference information on the basis of a difference between a first observation value corresponding to the first observation condition and a second observation value corresponding to the second observation condition.
(15) The information processing apparatus according to any one of (1) to (14), further including
   a plan generation unit that generates an imaging plan for acquiring the captured image as training data to be used for machine learning.
(16) The information processing apparatus according to (15), further including
   a presentation unit that presents the imaging plan to a user, in which
   the presentation unit presents a graphical user interface (GUI) in which the imaging plan is displayed so as to be identifiable to the user.
(17) An information processing method, including
   by a computer system
   generating difference information relating to a difference between a first observation condition that is an observation condition when observing an eye to be examined by a slit lamp microscope and a second observation condition that is an observation condition that is a basis with respect to observation of the eye to be examined by the slit lamp microscope.
(18) A program that causes a computer system to execute
   a step of generating difference information relating to a difference between a first observation condition that is an observation condition when observing an eye to be examined by a slit lamp microscope and a second observation condition that is an observation condition that is a basis with respect to observation of the eye to be examined by the slit lamp microscope.
(19) An ophthalmic microscope system, including:
   a slit lamp microscope; and
   an information processing apparatus including
      a generation unit that generates difference information relating to a difference between a first observation condition that is an observation condition when observing an eye to be examined by a slit lamp microscope and a second observation condition that is an observation condition that is a basis with respect to observation of the eye to be examined by the slit lamp microscope.

### REFERENCE SIGNS LIST

1 slit lamp microscope
2 illumination optical system
3 imaging optical system
12 image analysis unit
13 observation condition estimation unit
14 imaging plan generation unit
15 guide information generation unit
40 guide display GUI
100 observation system

1. An information processing apparatus, comprising
a generation unit that generates difference information relating to a difference between a first observation condition that is an observation condition when observing an eye to be examined by a slit lamp microscope and a second observation condition that is an observation condition that is a basis with respect to observation of the eye to be examined by the slit lamp microscope.

2. The information processing apparatus according to claim 1, further comprising
an estimation unit that estimates the observation condition on a basis of a captured image including the eye to be examined.

3. The information processing apparatus according to claim 1, wherein
the observation condition at least includes an illumination condition relating to an illumination optical system included in the slit lamp microscope and an imaging condition relating to an imaging optical system included in the slit lamp microscope.

4. The information processing apparatus according to claim 2, wherein
the estimation unit estimates the illumination condition on a basis of the captured image.

5. The information processing apparatus according to claim 3, wherein
the illumination condition includes at least one of a position, an illumination direction, an amount of light, or a shape of illumination light.

6. The information processing apparatus according to claim 2, wherein
the estimation unit estimates the imaging condition on a basis of the captured image.

7. The information processing apparatus according to claim 3, wherein
the imaging condition includes at least one of a position, a scale, or an imaging direction.

8. The information processing apparatus according to claim 3, wherein
the generation unit generates the difference information on a basis of a difference between a first illumination condition included in the first observation condition and a second illumination condition included in the second observation condition.

9. The information processing apparatus according to claim 3, wherein
the generation unit generates the difference information on a basis of a difference between a first imaging condition included in the first observation condition and a second imaging condition included in the second observation condition.

10. The information processing apparatus according to claim 1, further comprising
a presentation unit that presents the difference information to a user.

11. The information processing apparatus according to claim 10, wherein
the presentation unit presents a graphical user interface (GUI) in which the difference information is displayed so as to be identifiable to the user.

12. The information processing apparatus according to claim 10, wherein
the presentation unit presents the difference information to the user by sound.

13. The information processing apparatus according to claim 10, wherein
the slit lamp microscope includes an image display unit, and
the presentation unit causes the image display unit to display the GUI.

14. The information processing apparatus according to claim 1, wherein
the generation unit generates the difference information on a basis of a difference between a first observation value corresponding to the first observation condition and a second observation value corresponding to the second observation condition.

15. The information processing apparatus according to claim 1, further comprising
a plan generation unit that generates an imaging plan for acquiring the captured image as training data to be used for machine learning.

16. The information processing apparatus according to claim 15, further comprising
a presentation unit that presents the imaging plan to a user, wherein
the presentation unit presents a graphical user interface (GUI) in which the imaging plan is displayed so as to be identifiable to the user.

17. An information processing method, comprising
by a computer system
generating difference information relating to a difference between a first observation condition that is an observation condition when observing an eye to be examined by a slit lamp microscope and a second observation condition that is an observation condition that is a basis with respect to observation of the eye to be examined by the slit lamp microscope.

18. A program that causes a computer system to execute
a step of generating difference information relating to a difference between a first observation condition that is an observation condition when observing an eye to be examined by a slit lamp microscope and a second observation condition that is an observation condition that is a basis with respect to observation of the eye to be examined by the slit lamp microscope.

19. An ophthalmic microscope system, comprising:
a slit lamp microscope; and
an information processing apparatus including
a generation unit that generates difference information relating to a difference between a first observation condition that is an observation condition when observing an eye to be examined by a slit lamp microscope and a second observation condition that is an observation condition that is a basis with respect to observation of the eye to be examined by the slit lamp microscope.
